(19) United States
(12) Patent Application Publication
(10) Pub. No.: US 2014/0131257 A1
(43) Pub. Date: May 15, 2014

(54) PREPARATORY HIGH PERFORMANCE LIQUID CHROMATOGRAPHIC (HPLC) SEPARATION TECHNIQUE FOR QUANTITATIVE FRACTIONATION OF HEAVY PETROLEUM STREAMS

(71) Applicant: EXXONMOBIL RESEARCH AND ENGINEERING COMPANY, Annandale, NJ (US)

(72) Inventors: Birbal CHAWLA, Cherry Hill, NJ (US); Cathleen YUNG, Phillipsburg, NJ (US)

(73) Assignee: ExxonMobil Research and Engineering Company, Annandale, NJ (US)

(21) Appl. No.: 13/674,185

(22) Filed: Nov. 12, 2012

(51) Int. Cl. B01D 11/04

(52) U.S. Cl. CPC B01D 11/0492
USPC 208/314

(57) ABSTRACT

A method and system for performing quantitative fractionation of a hydrocarbon sample is disclosed. The method includes introducing the hydrocarbon sample in a separation system having a first separation column and a second separation column. Separate fraction removal steps are performed for the elution of saturates, aromatic ring classes, sulfides, and polars fractions. The method allows to collect the separated fractions and obtain the material balance information of the hydrocarbon sample. The method further includes performing an analysis on the at least one aromatic ring class fraction to identify and quantify an olefins fraction in the hydrocarbon sample.

### FIELD

The disclosed subject matter relates to a method and system for analyzing hydrocarbon containing oils. In particular, the disclosed subject matter relates to a method and system for conducting chromatographic analysis of hydrocarbon containing oils to provide quantification of eight classes of compounds (i.e., saturates, 1-4+ ring aromatics, sulfides, polars and olefins) contained within the hydrocarbon containing oils.

### BACKGROUND

Compositional analysis of heavy crude oils is essential to improve efficiency of refinery operations. The compositional analysis helps identify specific refineries that can process specific grades or classes of crude oil. The compositional analysis will also help identify what product slates are capable of being produced from a specific crude oil and what processing is required to create the product slate. These heavy crude oils are often too complex to be analyzed for detailed compositional information using most current analytical techniques/instruments. It is, therefore, essential to fractionate them into sub-groups of different classes of compounds so that the detail/extensive molecular compositional analyses can be performed using characterizing tools, e.g. high resolution mass spectroscopy.

Preparative liquid chromatography (LC) has been used very extensively to fractionate heavy streams in terms of mainly three classes of compounds: saturates, aromatics, and polars. Saturates include of n-paraffins, iso-paraffins, and naphthenes. Aromatics include aromatic-hydrocarbons, aromatic-thiophenes, and some sulfides. Polars include heteroatom containing complex organic compounds. Additionally, aromatics are further fractionated, using preparative liquid chromatography, mostly into four classes of aromatic compounds based upon number of aromatic rings such as one-, two-, three-, and four+ rings. Although these preparative liquid chromatographic approaches provide relatively pure fractions and the weight percent data, the LC separations are very laborious and time consuming and hence costly. These separations are not environmental friendly because they use large volume of organic solvents which must be evaporated in order to get pure fractions.

In the petroleum industry, the heavy petroleum streams (boiling above 550 degrees F.) are most often fractionated by two or more liquid chromatographic techniques in order to obtain detail compositional information. The accurate detail compositional information is essential in order to process the heavy streams (e.g. vacuum gas oil stream, etc.) into more profitable commodities, e.g. naphtha, diesel, etc. Additionally, detail characterization of heavy petroleum streams is required for developing new processing capabilities.

High Definition Hydrocarbon Analysis (HDHA) liquid chromotagraphic protocols for fractionating vacuum gas oils were developed about two decades ago and have been upgraded from time-to-time. Over the years, improvements have been made, such as the replacement of an open-glass-columns LC separation (i.e., silica gel separation) with reusable stainless steel columns. The open-column LC was replaced with an automated high performance liquid chromatography (HPLC) protocol. The HDHA laboratory prepared silver nitrate loaded non-reusable column was replaced with a reusable silver-ion column to retain sulfides and to achieve a base-line separation between saturates and one ring aromatics. The existing two high performance liquid chromatography (HPLC) separation techniques used for fractionation of vacuum gas oils are very time consuming and slow in providing HDHA data. One of the HPLC techniques, the Aromatic Ring Class (ARC) technique was developed about 20 years ago is operated at −40° C. and is hard to maintain. Another HPLC technique, the Silica Gel Separation (SGS) technique was developed about 10 years ago and is based upon packed HPLC columns. These two techniques use about 10-15 times more solvent volume (11.5 L Vs 0.70 L per sample) compared to the presently disclosed subject matter and takes about 20-25 hrs to complete one sample separation.

Most of these LC separations are performed at preparative scale so that enough of each of the separated fractions is obtained to perform other analyses. These separations are very cumbersome and mostly use silica gel or alumina as the stationary phase. The functionalized-silica-gel solid phases with amino- and/or cyano-groups have also been used. However, these separations provide some limited information on selected classes, namely saturates, aromatics, polars, and sub-fractions of aromatics.

Although the HDHA protocols were automated, these protocols were very time consuming. Commonly assigned U.S. Pat. No. 8,114,678 to Chawla et al discloses an automated analytical HPLC method for rapid quantitative determination of seven classes of compounds present in heavy petroleum streams boiling between 550° F. and 1050° F. that offers a significant improvement over the existing protocols. The seven classes of compounds are: saturates, aromatic-ring-classes 1-4, sulfides, and polars. The protocol disclosed by Chawla et al is referred to as STAR-7 (Synthesis TARget of 7 classes of compounds). The disclosure of U.S. Pat. No. 8,114,678 is incorporated herein in its entirety by reference. Type of analysis relates to the compositional analysis of both refinery and research samples. The synthesis in STAR-7 protocol refers to a data reconciliation procedure in which a detailed model-of-composition is adjusted to match analytical test results referred to as targets. The STAR-7 protocol provides seven analytical test results that are used in the reconciliation process. The STAR-7 protocol may be employed as part of the analytical protocol used in developing a model of composition for a hydrocarbon sample. Furthermore, the STAR-7 protocol can provide targets to which a reference model-of-composition is reconciled in estimating a model-of-composition for a sample under test. The analytical STAR-7 separation protocol utilizes two reusable columns and an HPLC system. The STAR-7 separation protocol and system offers an improvement over existing protocols because it can be performed in significantly less time (i.e., several days vs. 8-10 hours) and weight percent data for all seven fractions is obtained based on the detector's calibration response. Unfortunately, the use of an evaporative light scattering detector (ELSD) results in the destruction of the seven separated fractions. As such, further analysis cannot be performed on the separated fractions.

The compositions of crude oils and refinery process streams need to be available in a timely manner in order to utilize the molecule management approach to increase profits for refining and supply. There is a need for relatively fast and robust preparatively HPLC technique for separation of VGO type samples into seven compositionally different fractions which are analyzed further for detail molecular composition. There is also a need for an HPLC technique that provides cleaner/superior fractions. There is also a need for a technique that obtains quantitation of an additional class of compounds (olefins) by analyzing the separated aromatic fractions.

### SUMMARY

The presently disclosed subject matter is an improvement over the analytical STAR-7 separation protocol. The presently disclosed subject matter utilizes larger columns which provide higher loading capacities. In the presently disclosed subject matter, all seven fractions are quantitatively collected for their weight percentage determinations. The fractions are recoverable, which permits the analysis of these samples using a variety of analytical tools. This analysis permits the identification of an additional class of compounds.

The presently disclosed subject matter is directed to a method of performing quantitative fractionation of a hydrocarbon sample. The hydrocarbon sample containing saturates fraction, at least one aromatic ring class fraction, sulfides fraction, polars fraction and olefins fraction. The method includes providing a hydrocarbon sample for analysis and introducing the hydrocarbon sample in a separation system having a first separation column and a second separation column. The hydrocarbon sample is preferably a heavy crude oil or fraction thereof having a boiling point in excess of 500° F. The first separation column is a DNAP column containing 2,4-dinitroanilino-propyl-silica gel. The second separation column contains a silver-ion-loaded-strong-cation-exchange-silica gel (Ag+SCX—).

The method includes performing a saturates fraction removal process in the first and second separation columns to extract a saturates fraction from the hydrocarbon sample. The saturates fraction is removed by passing the hydrocarbon sample and a first solvent mixture through the first and second separation columns to elute the saturates. The solvent mixture contains hexane.

The method further includes performing an aromatic ring class fraction removal process in the second separation column to extract at least one aromatic ring class fraction from the hydrocarbon sample. This process is repeated four times in order to remove aromatic ring class 1-4+ fractions. After the aromatic ring class-1 fraction has been moved from the first column to the second column, it is removed by passing a second solvent mixture through the second separation column to elute the aromatic ring class-1 fraction from the hydrocarbon sample. The composition of the second solvent mixture varies over time with second solvent mixture initially contains a mixture of hexane, methylene chloride and toluene. The aromatic ring class-2 fraction is removed by passing the hydrocarbon sample and a third solvent mixture through the second separation column to elute the aromatic ring class-2 fraction. Like the second solvent, the composition of the third solvent mixture varies over time with third solvent mixture initially contains a mixture of hexane, methylene chloride and toluene. The aromatic ring class-3 fraction is removed by passing the hydrocarbon sample and a fourth solvent mixture through the second separation column to elute the aromatic ring class-3 fraction from the hydrocarbon sample. The fourth solvent mixture varies over time. The fourth solvent mixture initially contains a mixture of methylene chloride and toluene, after a predetermined time period the mixture is replaced with hexane. The aromatic ring class-4 fraction is removed by passing the hydrocarbon sample and a fifth solvent mixture through the second separation column to elute the aromatic ring class-4 fraction. The fifth solvent mixture initially contains a mixture of methylene chloride and toluene, after a predetermined time period the mixture is replaced with hexane. During all of the elution steps for the aromatic ring classes, each of the fractions is initially moved from the first column and then is eluted from the second column.

The method further includes performing a sulfides fraction removal process in the second separation column to extract a sulfides fraction from the hydrocarbon sample. After the saturates and aromatics removal, the sulfides fraction is removed by backflushing the second separation column with a sixth solvent mixture containing methylene chloride, toluene, and methanol.

The method further includes performing a polars fraction removal process in the first separation columns to extract a polars fraction from the hydrocarbon sample. The polars fraction is removed by backflushing the first separation column using a seventh solvent mixture. The seventh solvent mixture varies over time, wherein seventh solvent mixture initially contains a mixture of methylene chloride and methanol, after a predetermined time period the mixture is replaced with hexane.

The further includes performing an analysis on the at least one aromatic ring class fraction to identify an olefins fraction in the hydrocarbon sample. Performing an analysis on the at least one aromatic ring class fraction includes analyzing each of the aromatic ring class-1, aromatic ring class-2, aromatic ring class-3, aromatic ring class-4 fractions. The analysis includes performing ¹H NMR analysis on each of the aromatic ring class fractions.

### BRIEF DESCRIPTION OF THE DRAWINGS

FIG. 1 is a schematic diagram of the fraction separation protocol in accordance with the presently disclosed subject matter.

FIG. 2 is a schematic diagram of the fraction separation system in a saturates fraction elution mode in accordance with the presently disclosed subject matter.

FIG. 3 is a schematic diagram of the fraction separation system in an ARC fraction elution mode in accordance with the presently disclosed subject matter.

FIG. 4 is a schematic diagram of the fraction separation system in a sulfides fraction backflushing mode in accordance with the presently disclosed subject matter.

FIG. 5 is a schematic diagram of the fraction separation system in a polars fraction backflushing mode in accordance with the presently disclosed subject matter.

### DETAILED DESCRIPTION

While the disclosed subject matter may be embodied in many different forms, reference will now be made in detail to specific embodiments of the disclosed subject, examples of which are illustrated in the accompanying drawings. This description is an exemplification of the principles of the disclosed subject and is not intended to limit the disclosed subject matter to the particular embodiments illustrated.

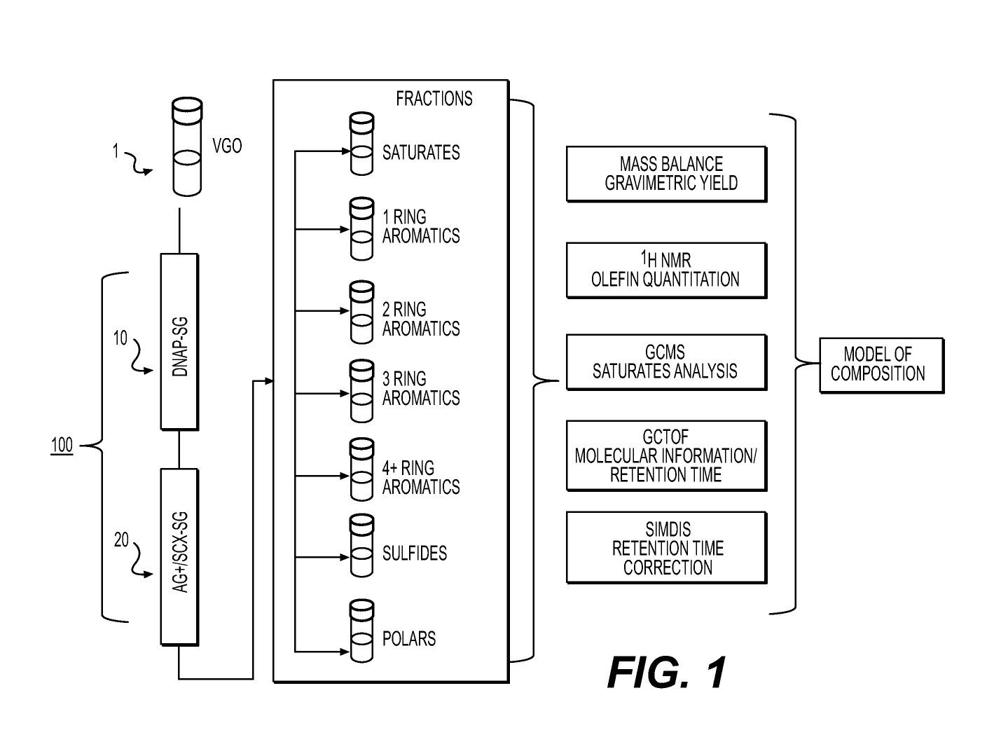

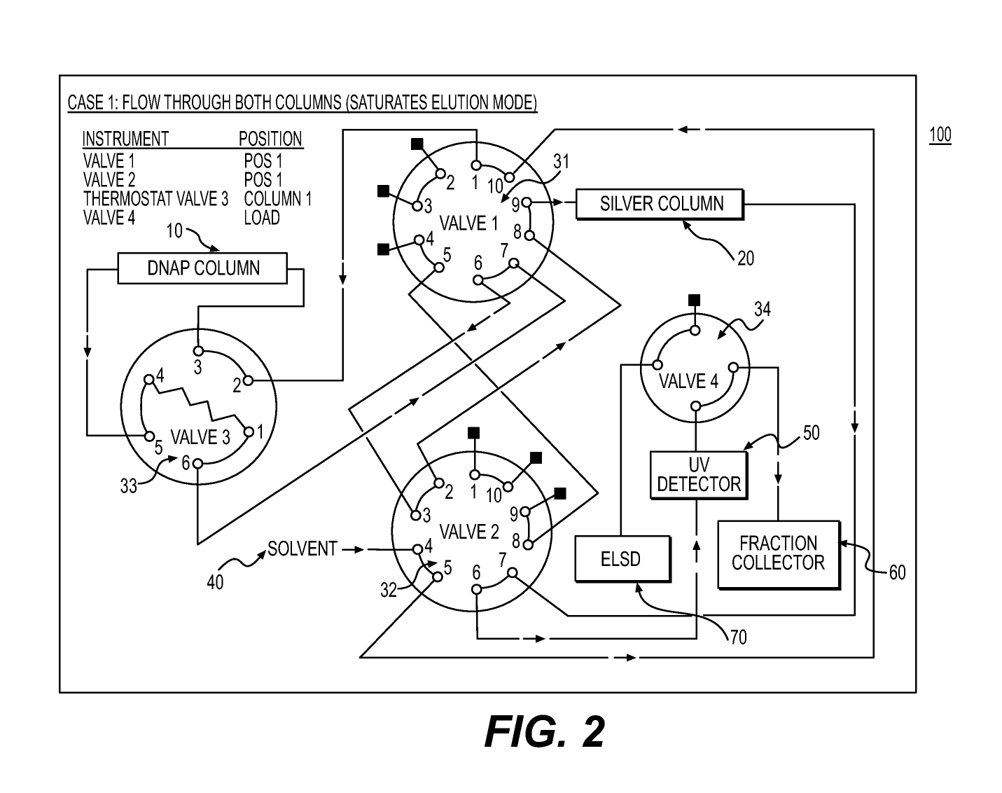

FIG. 1 is a schematic diagram illustrating the fraction separation and testing protocol in accordance with the presently disclosed subject matter. A hydrocarbon sample 1 is introduced into a separation system 100, which includes a pair of columns 10 and 20. The first column 10 is a DNAP column containing 2,4-dinitroanilino-propyl-silica gel. The second column 20 is a silver-ion-loaded-strong-cation-exchange-silica gel (Ag⁺SOC⁻). The first column 10 and the second column 20 are connected through switching valves (FIGS. 2-5). The separation system 100 separates seven fractions (i.e., saturates, 1-4+ ring class aromatics, sulfides and polars). The seven fractions are then subject to additional analysis to obtain more compositional details. The additional analysis may include but is not limited to mass balance, NMR, GCMS, GC/TOF and SIMDIS analysis. GCMS analysis is performed on the saturates fraction to obtain molecular type speciation (paraffins vs, naphthenes). GC/TOF and SIMDIS analyses are performed on the separated fractions in order to obtain molecular information. The combination of all the analyses (¹H NMR, GCMS, GC/TOF and SIMDIS) provide compositional information of the sample and therefore can be used to develop a model of composition. Mass balance analysis is used to obtain gravimetric yields by physically weighing the separated fractions. This non-destructive technique when compared to conventional ELSD analysis permits further analysis, which allows for the quantitation of an additional fraction (olefins).

An olefins fraction can be obtained by analyzing the 1-4 ring class aromatic fractions obtained during the initial fraction separation protocol. ¹H NMR spectroscopy compositional analysis is performed on the 1-4⁺ ring class aromatic fractions. The weight percent of the olefins in the fraction is calculated from an ¹H NMR integration of the fraction and average carbon number of the sample. The average carbon number is calculated from the carbon distribution of saturate fraction gas chromatogram or estimation from the type of hydrocarbon sample. This calculation of weight % of olefins assumes that the contribution of tetra-substituted olefins is negligible and that all of the olefins are aliphatic molecules with a single double bond in the molecule. In the ¹H NMR spectrum, there are five distinctive resonances associated with different types of olefin protons: 6.0-5.6 (A), 5.6-5.2 (B), 5.2-5.0 (C), 5.0-4.8 (D) and 4.8-4.6 (E) ppm. All of the signals from the ¹H NMR spectrum are intregrated and the integrals are normalized to 100. The integrals are then input into the following equation to calculate the weight % of the olefin fraction:

Wt % olefins=Σ[(int of C)/(1/2×average carbon#)]+

[(int of B+E)/(2/2×average carbon#)]+[(int of A+D)/(3/2×average carbon #)]

GC/TOF analysis is performed on the fractions obtain quantitation of the sulfur species present in the various fractions.

The results of the analysis including the quantitation of the olefins fraction can be used for several purposes. The analysis can be used to develop a model of composition, which can be used as a basis for identifying and predicting the properties of crude oils. The results can be used to identify the particular refinery or processing facility is best suit for processing the hydrocarbon and identify if any adjustments need to be made to operating conditions of the refinery to efficiently process the hydrocarbon. The results can also be used to identify the product slate that can be obtained from the particular hydrocarbon.

The system and process for separating seven fractions from the hydrocarbon sample in accordance with the presently disclosed subject matter will now be described in greater detail. The separation system 100 for use in connection with the presently disclosed subject matter utilizes two relatively larger high performance liquid chromatography columns compared to the columns disclosed in U.S. Pat. No. 8,114,678 (250 mm×10 mm compared to 250 mm×4.6 mm). The larger columns provide higher loading capacity (100 mg compared to 0.2 mg). The first column 10 is a DHAP column containing 2,4-dinitroanilino-propyl-silica gel. The second column 20 is a silver-ion-loaded-strong-cation-exchange-silica gel (Ag⁺SCX⁻). The first column 10 and the second column 20 are connected through switching valves.

Switching valves 31, 32 and 33 are provided to control the flow of solvent and samples through the first and second columns. The switching valves 31, 32 and 33 are operatively couple to each other to control and adjust the flow of solvent and samples through the first column 10 and the second column 20. The first switching valve 31 is operatively connected to the second column 20, the second switching valve 32 and the third switching valve 33. The second switching valve 32 is operatively coupled to the first switching valve 31, the second column 20, a solvent supply unit 40 and a UV detector 50. The third switching valve 33 is operatively to the first switching valve 31 and the first column 10. A fourth switching valve 34 is operatively connected to the UV detector 50, a fraction collector 60 and an evaporative light scattering detector (ELSD) 70.

The first switching valve 31 and the second switching valve 32 are ten-port switching valves. The third switching valve 33 is a thermostat six-port switching valve. The fourth switching valve 34 is a four-port switching valve.

The solvent supply unit 40 is programmed to deliver four different solvents and mixtures thereof in a selected ratio for a specified duration at a desired rate. The solvents include hexane, methylene chloride, methanol and toluene. The desired rate is up to 10 ml/min.

During the fraction separation protocol in connection with the presently disclosed subject matter, the first switching valve 31, the second switching valve 32 and the third switching valve 33 are switched between first and second positions to control the flow of sample and solvent through the first and second columns 10 and 20 to facilitate separation of each of the seven fractions. The mixture of solvents delivered by the solvent supply unit 40 for each of the fractions separated may vary.

The elution of seven classes of compounds will now be described in greater detail in connection with the figures.

The elution of saturates from a vacuum gas oil (VGO) hydrocarbon sample will be described in connection with FIG. 2. The schedule of solvents disclosed herein is suitable for the extraction of fractions from a VGO sample. It is contemplated that other mixtures of solvents may be used in connection with the separation of fractions for other hydrocarbon samples. FIG. 2 illustrates the saturates elution mode for the system 100. In the saturates elution mode, the saturates fraction is eluted from the sample and sample containing the ARC-1 fraction is moved to the second column 20. In the saturates elution mode, the first switching valve 31 and the second switching valve 32 are in a first position. The third thermostat valve 33 is in a first column position. With such an arrangement, the hydrocarbon sample and the predetermined solvents are pass through both the first column 10 and the second column 20. The solvent from the solvent supply unit 40 is fed into the system 100 through the second switching valve 32. The solvent and the sample pass through the first switching valve 31 to the third thermostat valve 33 to the first column 10. The solvent and sample pass through the first column 10 in a first direction as indicated by the arrow in FIG. 2. The solvent and sample mixture then pass through the third thermostat valve 33 to the first switching valve 31 to the second switching valve 32 and back to the first switching valve 31 before it passes through the second column 20 in the direction indicated by the arrow in FIG. 2. From the second column 20, the eluded saturates fraction pass from the second column 20 to the second switching valve 32 and then to the UV detector 50. The fourth switching valve 34 is in a load position such that the saturates fraction passes through the valve 34 to the fraction collector 70. In the saturation elution mode, the solvent supply unit 40 supplies only hexane into the system at the flow rate indicated in Table 1.

TABLE 1
Elution Steps and Solvent Supply Unit Deliver Schedule

| | | | Methylene | | | |
|---|---|---|---|---|---|---|
| | Time | Hexane | Chloride | Methanol | Toluene | Flow |
| Elution Step | (min) | (%) | (%) | (%) | (%) | (mL/min) |
| Eluting Saturates and Moving | 0.01 | 100 | 0 | 0 | 0 | 10.0 |
| ARC-1 to Ag⁺/SCX⁻ | 7.00 | 100 | 0 | 0 | 0 | 10.0 |
| Eluting ARC-1 and Moving | 7.05 | 68 | 30 | 0 | 2 | 10.0 |
| ARC-2 to Ag⁺/SCX⁻ | 8.00 | 70 | 30 | 0 | 0 | 10.0 |
| | 8.05 | 100 | 0 | 0 | 0 | 10.0 |
| | 15.00 | 100 | 0 | 0 | 0 | 10.0 |
| Eluting ARC-2 | 15.05 | 65 | 30 | 0 | 5 | 10.0 |
| | 19.00 | 70 | 30 | 0 | 0 | 10.0 |
| | 19.05 | 100 | 0 | 0 | 0 | 10.0 |
| | 24.00 | 100 | 0 | 0 | 0 | 10.0 |
| Moving ARC-3 to Ag⁺/SCX⁻ | 24.05 | 80 | 20 | 0 | 0 | 10.0 |
| | 28.00 | 90 | 10 | 0 | 0 | 10.0 |
| Eluting ARC-3 | 28.05 | 0 | 85 | 0 | 15 | 10.0 |
| | 31.50 | 0 | 100 | 0 | 0 | 10.0 |
| | 31.55 | 100 | 0 | 0 | 0 | 10.0 |
| | 33.00 | 100 | 0 | 0 | 0 | 10.0 |
| Moving ARC-4⁺ to Ag⁺/SCX⁻ | 33.05 | 40 | 60 | 0 | 0 | 10.0 |
| and Eluting ARC-4⁺ | 48.00 | 0 | 36 | 4 | 60 | 10.0 |
| Backflushing Sulfides | 48.05 | 0 | 40 | 10 | 50 | 10.0 |
| | 60.00 | 0 | 40 | 10 | 50 | 10.0 |
| Backflushing Polars | 60.05 | 0 | 90 | 10 | 0 | 10.0 |
| | 63.00 | 0 | 90 | 10 | 0 | 10.0 |
| | 63.05 | 0 | 100 | 0 | 0 | 10.0 |
| | 67.00 | 0 | 100 | 0 | 0 | 10.0 |
| System Cleaning | 67.00 | 0 | 100 | 0 | 0 | 10.0 |
| | 80.00 | 0 | 100 | 0 | 0 | 10.0 |

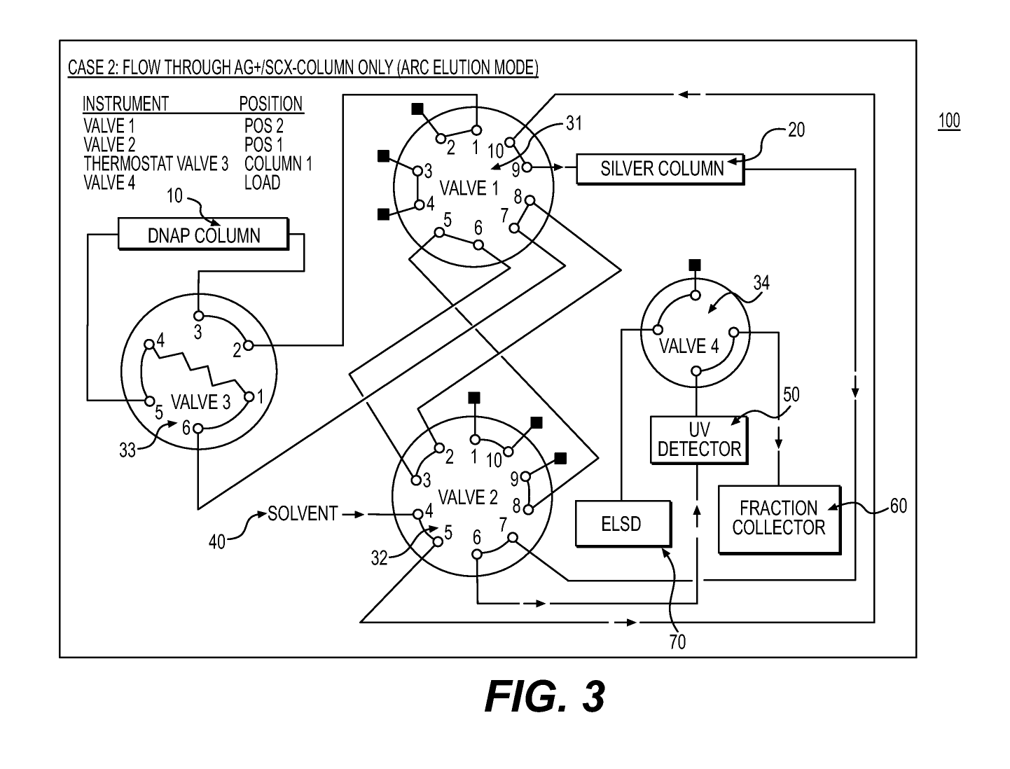

The elution of the aromatic ring class (ARC) fractions will be described in connection with FIG. 3. FIG. 3 illustrates the ARC fraction elution mode for the system 100. In the ARC fraction elution mode, the ARC fractions are eluted from the sample. In the ARC fraction elution mode, only the second column 20 is used. The first column 10 is by-passed after the ARC fraction has been moved from the first column 10 to the second column 20. This is accomplished by moving the first switching valve 31 to a second position. The second switching valve 32 remains in the first position. The third thermostat valve 33 remains in the first column position. With such an arrangement, the remaining hydrocarbon sample and the predetermined solvents pass only through the second column 20 so the desired ARC fraction can be eluted. The solvent from the solvent supply unit 40 is fed into the system 100 through the second switching valve 32. The solvent then passes through the first switching valve 31 to the second column 20 in the first direction as indicated by the arrow in FIG. 3. From the second column 20, the eluded ARC fraction passes from the second column 20 to the second switching valve 32 and then to the UV detector 50. The fourth switching valve 34 is in a load position such that the eluted ARC fraction passes through the valve 34 to the fraction collector 70. In the ARC fraction elution mode, the solvent supply unit 40 supplies a varying mixture of solvents, as illustrated in Table 1.

As illustrated in Table 1, the mixture of solvent varies during the elution of the 1-Ring Aromatics or ARC-1 fraction. Initially, a mixture of hexane (68%), methylene chloride (30%) and toluene (2%) is supplied. The amount of hexane (70%) is increased, while the supply of toluene (0%) is eliminated. The supply of solvent is then increased to 100% hexane for remainder of the ARC-1 fraction elution. In the ARC-1 fraction elution step, the ARC-1 fraction is removed from the second column 20 to the fraction collector 70 and the 2-Ring Aromatics or ARC-2 fraction is moved to the second column 20 for elution.

As illustrated in Table 1, the mixture of solvent varies during the elution of the 2-Ring Aromatics or ARC-2 fraction. Initially, a mixture of hexane (65%), methylene chloride (30%) and toluene (5%) is supplied. The amount of toluene is greater than the amount used in the ARC-1 fraction elution. Overtime, the amount of hexane (70%) is increased, while the supply of toluene (0%) is eliminated. The supply of solvent is then increased to 100% hexane for remainder of the ARC-2 fraction elution. In the ARC-2 fraction elution step, the ARC-2 fraction is removed from the second column 20 to the fraction collector 70.

In preparation for the elution of the 3-Ring Aromatics or ARC-3 fraction, the switching valves are positioned as in FIG. 2 and a mixture of hexane (80%) and methylene chloride (20%) is supplied from the solvent supply unit 40. Overtime, the amount of hexane (90%) is increased, while the supply of methylene chloride (10%) is reduced. This allows the ARC-3 to move from the first column 10 to the second column 20.

After the ARC-3 has moved to the second column 20, the switching valves are re-positioned as in FIG. 3. The mixture of solvent varies during the elution of the 3-Ring Aromatics or ARC-3 fraction. Initially, a mixture of methylene chloride (85%) and toluene (15%) is supplied. The amount of methylene chloride (100%) is increased, while the supply of toluene (0%) is eliminated. The supply of solvent is then changed from 100% methylene chloride to 100% hexane for remainder of the ARC-3 fraction elution. In the ARC-3 fraction elution step, the ARC-3 fraction is removed from the second column 20 to the fraction collector 70.

In preparation for the elution of the 4-Ring Aromatics or ARC-4 fraction and its subsequent elution, a mixture of hexane (40%) and methylene chloride (60%) is supplied from the solvent supply unit 40 to move the ARC-4 fraction to the second column 20 using switching valve arrangement as shown in FIG. 2. After the ARC-4 has been moved to the second column 20, the switching valves are re-positioned as shown in FIG. 3 and the solvent mixture is then changed to a mixture of methylene chloride (36%), methanol (4%) and toluene (60%). The ARC-4 fraction is removed from the second column 20 to the fraction collector 70.

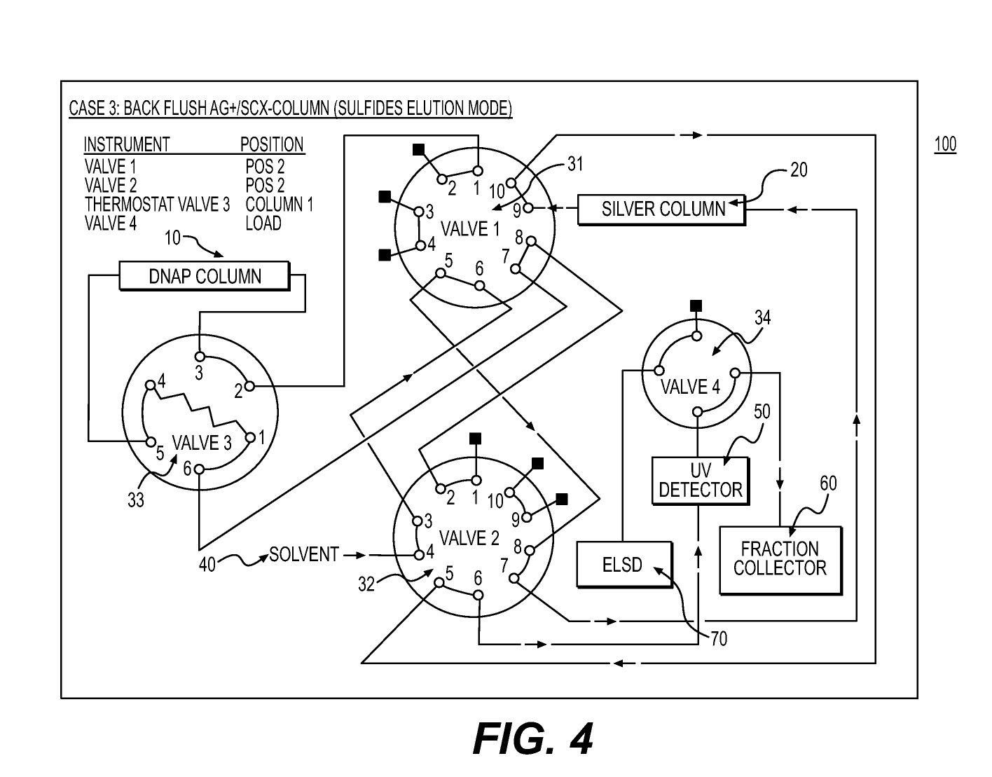

The removal of the sulfides fraction will be described in connection with FIG. 4. The elution of the sulfides fraction is accomplished by backflushing the second column 20. FIG. 4 illustrates the sulfides backflushing mode for the system 100. In the sulfides backflushing mode, the concentrated sulfides fraction is removed from the second column 20. The first column 10 is again by-passed. This is accomplished by maintaining the first switching valve 31 in the second position and moving the second switching valve 32 to a second position. The third thermostat valve 33 remains in the first column position. With such an arrangement, the predetermined solvents pass only through the second column 20 but in an opposite direction when compared to elution of the ARC fractions. The solvent from the solvent supply unit 40 is fed into the system 100 through the second switching valve 32. The solvent then passes through the first switching valve 31 back through the second switching valve 32 to the second column 20 in the second direction, as indicated by the arrow in FIG. 4. From the second column 20, the backflushed sulfides pass from the second column 20 to the second switching valve 32 and then to the UV detector 50. The fourth switching valve 34 is in a load position such that the eluted sulfides fraction passes through the valve 34 to the fraction collector 70. In the backflushing sulfides mode, the solvent supply unit 40 supplies a mixture of solvents that does not vary during the backflushing. The mixture contains methylene chloride (40%), methanol (10%) and toluene (50%).

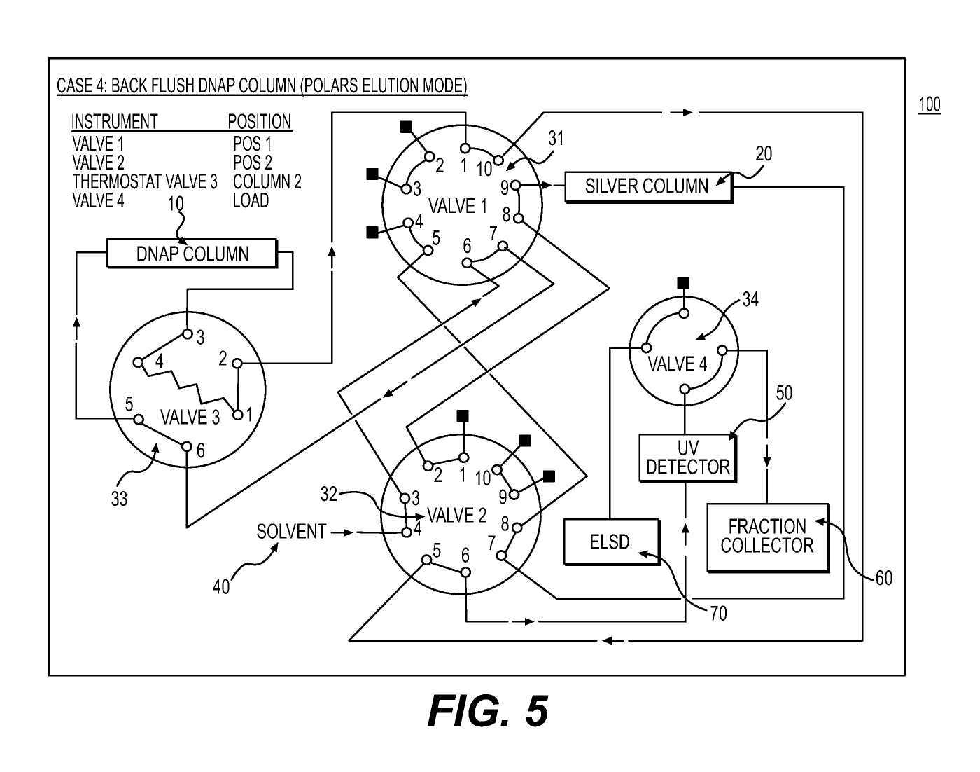

The removal of the polars fraction will be described in connection with FIG. 5. The elution of the polars fraction is accomplished by backflushing the first column 10. FIG. 4 illustrates the polars backflushing mode for the system 100. In the polars backflushing mode, the polars fraction is removed from the first column 10. The second column 20 is now by-passed. This is accomplished by moving the first switching valve 31 from the second position back to the first position and maintaining the second switching valve 32 in the second position. The third thermostat valve 33 is switched to a second column position. With such an arrangement, the remaining hydrocarbon sample and the predetermined solvents pass only through the first column 10 but in an opposite direction when compared to elution of the saturates fraction. The solvent from the solvent supply unit 40 is fed into the system 100 through the second switching valve 32. The solvent then passes through the first switching valve 31 to third thermostat valve 33 to the first column 10 in the second direction, as indicated by the arrow in FIG. 5. From the first column 10, the backflushed polars fraction pass from the first column 10 to the first switching valve 31 to the second switching valve 32 and then to the UV detector 50. The fourth switching valve 34 is in a load position such that the eluted polars fraction passes through the valve 34 to the fraction collector 70. In the backflushing polars mode, the solvent supply unit 40 supplies a mixture of solvents that varies during the backflushing. The mixture contains methylene chloride (90%) and methanol (10%). The methylene chloride content is then increased to 100%.

As evidenced by the time indicator in Table 1, the elution of the seven fractions can be accomplished in slightly over an hour. This is a significant decrease in time when compared to the prior art HDHA protocols.

Following the completion of the removal of the fractions, the system 100 is returned to the configuration illustrated in FIG. 2 such that the system can be cleaned with the exception that the flow from the second switching valve 32 is diverted from the UV detector. Instead, the flow of solvent is diverted to a post cleaning receptable (not shown). During the system cleaning, the solvent supply unit 100 introduces only methylene chloride into the system. After cleaning, the system 100 is re-generated using 100% hexane so that the system becomes ready to elute fractions from another hydrocarbon sample.

In order to establish the repeatability/reproducibility of the measurements obtained from the fraction elution steps disclosed above, seven runs were made using a heavy petroleum distillate solution in cyclo-hexane. The protocol was run in the fraction collection modes illustrated in FIGS. 2-5. A set of two runs was made for every sample analyzed for good mass recovery as well as for further detail analyses to be performed on the fractions.

The average values (wt %) for each of the seven fractions from all the seven runs along with the corresponding known HDHA values for the same hydrocarbon sample are provided in Table 2. As shown by the data in Table 2, although there are small differences between the ARC-4 and sulfides fractions average values, the values obtained in accordance with the presently disclosed subject matter compared very well with the average HDHA values that have be determined over many years of testing. Table 2 clearly demonstrates that the accuracy along with precision and repeatability of the presently disclosed subject matter is consistent with those of HDHA.

TABLE 2
Comparison of Fractions Obtained from Current Separation Protocol
and Prior Art HDHA Fractions for Heavy Petroleum Distillate

| | | ARC- | ARC- | ARC- | ARC- | | |
|---|---|---|---|---|---|---|---|
| Run # | Sats | 1 | 2 | 3 | 4 | Sulfides | Polars |
| 1 | 48.5 | 15.7 | 14.7 | 9.8 | 5.1 | 4.3 | 1.8 |
| 2 | 48.5 | 15.3 | 14.7 | 9.8 | 4.9 | 4.5 | 2.3 |
| 3 | 48.7 | 15.7 | 14.1 | 10.4 | 5.0 | 4.6 | 1.6 |
| 4 | 49.3 | 16.6 | 14.1 | 9.6 | 4.7 | 4.1 | 1.5 |
| 5 | 48.9 | 16.7 | 13.6 | 10.1 | 4.8 | 4.3 | 1.7 |
| 6 | 48.3 | 16.0 | 14.0 | 10.4 | 4.8 | 4.2 | 1.3 |
| 7 | 48.5 | 15.5 | 14.5 | 10.2 | 4.9 | 4.9 | 1.5 |
| Average | 48.7 | 16.0 | 14.2 | 10.0 | 4.9 | 4.3 | 1.7 |
| Standard | 0.3 | 0.5 | 0.4 | 0.3 | 0.1 | 0.3 | 0.3 |
| Deviation | | | | | | | |
| HDHA, Average | 48.3 | 15.9 | 14.1 | 10.6 | 5.8 | 4.0 | 1.3 |
| Standard | 0.6 | 0.7 | 0.6 | 0.6 | 0.4 | 0.4 | 0.2 |
| Deviation | | | | | | | |

In order to further validate the protocol and system for performing the same in accordance with the presently disclosed subject matter, a set of twenty five (25) different hydrocarbon samples that had been previously analyzed using conventional HDHA techniques were separated into fractions utilizing the current separation protocol of the presently disclosed subject matter. The hydrocarbon samples represent a wide range of sulfur (0.05-3.64%), saturates (19.5-94.1%), aromatics (5.0%-74.4%) and sulfides (0.6-0.9) contents. The samples included whole crude oils, vacuum gas oil blends, distillates, raffinates and extracts. The fractions obtained from the current separation protocol were mass balanced and compared with the values previously obtained using the conventional HDHA techniques. The results of the comparison are summarized in Tables 3a and 3b.

TABLE 3a
Comparison of Fractions Obtained from Current Separation
Protocol and Prior Art HDHA Fractions

| | Separation | | | | | | | | Total % |
|---|---|---|---|---|---|---|---|---|---|
| Description | Type | Sats | ARC1 | ARC2 | ARC3 | ARC4 | Sulfides | Polars | Recovery |
| Crude 1 | HDHA | 45.4 | 12.2 | 11.8 | 11.5 | 9.4 | 8.1 | 1.6 | |
| | Current | 45.7 | 11.5 | 15.3 | 10.5 | 6.9 | 7.7 | 2.3 | 94.1 |
| Crude 2 | HDHA | 31.9 | 17.9 | 11.4 | 11.3 | 8.6 | 10.1 | 8.8 | |
| | Current | 30.5 | 16.0 | 15.0 | 10.3 | 10.9 | 7.3 | 9.9 | 95.4 |
| Blend 1 | HDHA | 58.9 | 14.5 | 10.0 | 7.8 | 4.5 | 2.2 | 2.1 | |
| | Current | 57.6 | 15.4 | 10.7 | 7.0 | 5.1 | 1.5 | 2.7 | 98.8 |
| Crude 3 | HDHA | 60.7 | 13.9 | 7.9 | 8.0 | 6.5 | 2.1 | 1.1 | |
| | Current | 60.9 | 16.5 | 10.0 | 6.0 | 3.6 | 1.7 | 1.4 | 97.7 |
| Crude 4 | HDHA | 42.3 | 14.3 | 17.1 | 11.3 | 4.9 | 8.7 | 1.3 | |
| | Current | 41.4 | 14.1 | 16.3 | 10.4 | 6.6 | 9.0 | 2.2 | 91.9 |
| Crude 5 | HDHA | 46.9 | 15.7 | 12.5 | 11.5 | 6.6 | 5.2 | 1.7 | |
| | Current | 46.9 | 15.1 | 13.2 | 10.1 | 6.7 | 5.3 | 2.7 | 89.6 |
| Blend 2 | HDHA | 47.3 | 14.1 | 12.5 | 8.8 | 6.8 | 7.2 | 3.3 | |
| | Current | 51.9 | 14.4 | 12.7 | 7.8 | 5.1 | 6.0 | 2.2 | 94.4 |
| Crude 6 | HDHA | 81.4 | 7.2 | 5.2 | 3.1 | 1.7 | 0.7 | 0.7 | |
| | Current | 81.5 | 6.9 | 5.7 | 2.7 | 1.8 | 0.7 | 0.8 | 92.6 |
| Blend 3 | HDHA | 82.9 | 6.0 | 4.2 | 3.1 | 2.2 | 0.7 | 0.9 | |
| | Current | 83.3 | 6.0 | 3.6 | 2.6 | 2.5 | 0.8 | 1.2 | 93.3 |
| Blend 4 | HDHA | 62.2 | 14.0 | 6.8 | 4.9 | 2.5 | 2.3 | 1.1 | |
| | Current | 60.3 | 18.2 | 9.8 | 4.9 | 3.0 | 2.8 | 1.0 | 94.7 |
| Crude 7 | HDHA | 94.5 | 2.9 | 0.7 | 0.4 | 0.3 | 0.6 | 0.4 | |
| | Current | 94.1 | 2.8 | 0.7 | 0.4 | 0.7 | 0.9 | 0.5 | 92.6 |
| Crude 8 | HDHA | 46.9 | 25.6 | 12.9 | 8.3 | 3.6 | 1.8 | 1.0 | |
| | Current | 46.2 | 28.5 | 13.3 | 6.5 | 3.0 | 1.3 | 1.2 | 93.5 |
| Crude 9 | HDHA | 45.1 | 12.9 | 16.8 | 11.6 | 4.6 | 7.6 | 1.4 | |
| | Current | 45.1 | 14.4 | 14.9 | 8.7 | 6.4 | 7.6 | 3.0 | 94.4 |

The results obtained from the current separation protocol compare quite well to the results obtained from the earlier HDHA analysis. Table 3a, above, and Table 3b, below, clearly demonstrate that the accuracy along with precision and repeatability of the presently disclosed subject matter is consistent with those of HDHA.

TABLE 3b
Comparison of Fractions Obtained from Current Separation
Protocol and Prior Art HDHA Fractions

| | Separation | | | | | | | | Total % |
|---|---|---|---|---|---|---|---|---|---|
| Description | Type | Sats | ARC1 | ARC2 | ARC3 | ARC4 | Sulfides | Polars | Recovery |
| Crude 10 | HDHA | 51.8 | 28.6 | 10.9 | 5.1 | 2.0 | 0.7 | 0.9 | |
| | Current | 52.3 | 29.1 | 10.6 | 4.7 | 1.8 | 0.7 | 0.9 | 90.3 |
| Diluted | HDHA | 30.4 | 15.9 | 17.7 | 13.9 | 5.9 | 9.6 | 5.4 | |
| Bitumen | Current | 30.4 | 17.8 | 14.9 | 11.5 | 8.5 | 9.8 | 7.1 | 94.8 |
| Crude 11 | HDHA | 50.0 | 18.4 | 11.6 | 9.3 | 4.2 | 2.6 | 2.8 | |
| | Current | 48.8 | 19.5 | 11.1 | 8.3 | 5.7 | 3.4 | 3.2 | 94.2 |
| Crude 12 | HDHA | 84.5 | 5.5 | 4.1 | 3.1 | 1.3 | 0.7 | 0.6 | |
| | Current | 84.2 | 6.5 | 3.5 | 2.6 | 1.9 | 0.7 | 0.6 | 96.4 |
| Distillate 1 | HDHA | 60.0 | 14.4 | 6.9 | 8.6 | 5.7 | 3.6 | 0.9 | |
| | Current | 57.6 | 14.7 | 9.7 | 8.7 | 5.0 | 3.2 | 1.1 | 96.4 |
| Extract 1 | HDHA | 20.3 | 16.5 | 16.8 | 23.1 | 15.3 | 6.1 | 1.9 | |
| | Current | 19.5 | 16.0 | 22.2 | 22.0 | 12.0 | 6.0 | 2.1 | 97.2 |
| Raffinate 1 | HDHA | 77.7 | 12.6 | 2.8 | 1.5 | 1.5 | 3.1 | 0.1 | |
| | Current | 79.0 | 13.7 | 3.7 | 0.8 | 0.7 | 1.6 | 0.4 | 95.9 |
| Distillate 2 | HDHA | 55.0 | 14.6 | 9.8 | 7.5 | 4.7 | 5.4 | 1.2 | |
| | Current | 55.7 | 16.5 | 11.2 | 7.3 | 4.3 | 3.8 | 1.3 | 98.3 |
| Extract 2 | HDHA | 32.7 | 18.9 | 17.9 | 14.5 | 8.4 | 5.6 | 2.0 | |
| | Current | 32.5 | 20.0 | 19.4 | 13.4 | 7.2 | 5.9 | 1.7 | 96.8 |
| Raffinate 2 | HDHA | 80.7 | 10.3 | 2.7 | 0.7 | 0.9 | 2.5 | 0.2 | |
| | Current | 80.6 | 12.6 | 3.6 | 0.7 | 0.8 | 1.3 | 0.4 | 97.9 |
| Lube | HDHA | 30.0 | 8.1 | 6.1 | 22.6 | 31.1 | 0.5 | 1.7 | |
| | Current | 30.0 | 7.3 | 9.8 | 27.3 | 21.7 | 0.6 | 3.2 | 98.0 |
| Coker Run | HDHA | 36.2 | 11.3 | 8.8 | 10.7 | 15.9 | 2.9 | 8.5 | |
| 500-900 F. | Current | 36.3 | 14.5 | 14.0 | 11.6 | 14.9 | 3.1 | 5.5 | 88.7 |

The presently disclosed subject matter permits are more rapid, high accurate analysis of the fractions composing a particular hydrocarbon sample. The testing protocol and its associated separation system result in a significant time savings which can be translated into improved efficiency in the refining process. Refinery operators are able to more readily and accurately adjust refinery operations to process the given hydrocarbon to produce a desired product slate.

The disclosed subject matter is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

What is claimed is:

1. A method of performing quantitative fractionation of a hydrocarbon sample, wherein the hydrocarbon sample containing saturates fraction, at least one aromatic ring class fraction, sulfides fraction, polars fraction and olefins fraction, comprising:
providing a hydrocarbon sample;
introducing the hydrocarbon sample in a separation system having a first separation column and a second separation column;
performing a saturates fraction removal process in the first and second separation columns to extract a saturates fraction from the hydrocarbon sample;
performing an aromatic ring class fraction removal process in the second separation column to extract at least one aromatic ring class fraction from the hydrocarbon sample;
performing a sulfides fraction removal process in the second separation column to extract a sulfides fraction from the hydrocarbon sample;
performing a polars fraction removal process in the first separation columns to extract a polars fraction from the hydrocarbon sample;
performing a weight percent recovery analysis of the separated seven fractions of the hydrocarbon sample; and
performing an analysis on the at least one aromatic ring class fraction to identify an olefins fraction in the hydrocarbon sample.

2. The method according to claim 1, wherein the hydrocarbon sample is a vacuum gas oil.

3. The method according to claim 1, wherein the first separation column is a DNAP column containing 2,4-dinitroanilino-propyl-silica gel.

4. The method according to claim 1, wherein the second separation column contains a silver-ion-loaded-strong-cation-exchange-silica gel (Ag⁺SCX⁻).

6. The method according to claim 1, wherein performing a saturates fraction removal process includes passing the hydrocarbon sample and a first solvent mixture through the first and second separation columns.

7. The method according to claim 6, wherein the solvent mixture contains hexane.

8. The method according to claim 1, wherein performing an aromatic ring class fraction removal process includes passing the hydrocarbon sample and a second solvent mixture through the second separation column to extract an aromatic ring class-1 fraction from the hydrocarbon sample.

9. The method according to claim 8, wherein the second solvent mixture varies over time, wherein second solvent mixture initially contains a mixture of hexane, methylene chloride and toluene.

10. The method according to claim 8, wherein performing an aromatic ring class fraction removal process includes passing the hydrocarbon sample and a third solvent mixture through the second separation column to extract an aromatic ring class-2 fraction from the hydrocarbon sample.

11. The method according to claim 10, wherein the third solvent mixture varies over time, wherein third solvent mixture initially contains a mixture of hexane, methylene chloride and toluene.

12. The method according to claim 10, wherein performing an aromatic ring class fraction removal process includes passing the hydrocarbon sample and a fourth solvent mixture through the second separation column to extract an aromatic ring class-3 fraction from the hydrocarbon sample.

13. The method according to claim 12, wherein the fourth solvent mixture varies over time, wherein third solvent mixture initially contains a mixture of methylene chloride and toluene, after a predetermined time period the mixture is replaced with hexane.

14. The method according to claim 12, wherein performing an aromatic ring class fraction removal process includes passing the hydrocarbon sample and a fifth solvent mixture through the second separation column to extract an aromatic ring class-4 fraction from the hydrocarbon sample.

15. The method according to claim 14, wherein the fourth solvent mixture varies over time, wherein third solvent mixture initially contains a mixture of methylene chloride and toluene, after a predetermined time period the mixture is replaced with hexane.

16. The method according to claim 14, wherein performing an analysis on the at least one aromatic ring class fraction includes analyzing each of the aromatic ring class-1, aromatic ring class-2, aromatic ring class-3, aromatic ring class-4 fractions.

17. The method according to claim 16, wherein analyzing include performing ¹H NMR analysis on each of the aromatic ring class fractions.

18. The method according to claim 17, further comprising calculating the weight percent of olefins present in the hydrocarbon sample based upon the ¹H NMR analysis.

19. The method according to claim 1, wherein performing a sulfides fraction removal process includes backflushing the hydrocarbon sample and a sixth solvent mixture through the second separation column.

20. The method according to claim 19, wherein the sixth solvent mixture contains a mixture of methylene chloride, methanol and toluene.

21. The method according to claim 1, wherein performing a polars fraction removal process includes backflushing the hydrocarbon sample and a seventh solvent mixture through the first separation columns.

22. The method according to claim 21, wherein the seventh solvent mixture varies over time, wherein seventh solvent mixture initially contains a mixture of methylene chloride and methanol, after a predetermined time period the mixture is replaced with hexane,
performing an analysis on the at least one aromatic ring class fraction to identify an olefins fraction in the hydrocarbon sample.

23. The method according to claim 1, wherein performing an analysis on the at least one aromatic ring class fraction includes performing ¹H NMR analysis on each of the aromatic ring class fractions.

24. The method according to claim 23, further comprising calculating the weight percent of olefins present in the hydrocarbon sample based upon the ¹H NMR analysis.
